(19) United States
(12) Patent Application Publication
(10) Pub. No.: US 2011/0130673 A1
(43) Pub. Date: Jun. 2, 2011

(54) Apparatus for determining respiratory muscle endurance of a person

(76) Inventor: Christopher Patrick Lawson, Kent (GB)

(21) Appl. No.: 10/563,891

(22) PCT Filed: Jul. 12, 2004
(86) PCT No.: PCT/GB04/03009

§ 371 Date: Dec. 4, 2008

(30) Foreign Application Priority Data
Jul. 11, 2003 (GB) 0316349.0

(51) Int. Cl. A61B 5/08

(52) U.S. Cl. 600/529

(57) ABSTRACT

Apparatus (2) for determining respiratory muscle endurance of a person, which apparatus (2) comprises a mouthpiece (4) through which the person inspires, load-providing means (6) for providing a pressure against which the person inspires, and pressure control means (8) for controlling the pressure, the pressure control means (8) being such that it controls the pressure in response to a breathing pattern of the person.

This invention relates to apparatus for determining respiratory muscle endurance of a person.

The respiratory muscle endurance of a person is important because it determines the person's susceptibility to developing serious breathing difficulties which may lead to respiratory failure and therefore death. The person's susceptibility to developing serious breathing difficulties is especially prevalent in persons with chronic lung disease, in whom impairment of respiratory muscle function often co-exists with disease of the person's lungs or airways.

There are known different types of apparatus for determining the respiratory muscle endurance of a person. These known types of apparatus comprise a mouthpiece through which the person inspires, and load-providing means for providing a pressure against which the person inspires. The endurance is measured as the duration for which respiration can be sustained by the person against the pressure. This pressure may be increased at set time intervals according to known and pre-defined protocols. The disadvantage of the known apparatus is that the work done against the pressure load, and hence the energy expended, depends upon the pattern of breathing of the person. This pattern of breathing tends to be unrepresentative of the person's usual pattern of breathing. This is because the imposition of the fixed pressure load by the load-providing means is something that the person is not used to. For example, the person's breathing pattern may vary if the load-producing means is a valve with an orifice which suddenly opens. Also for example, the person's breathing pattern may depend upon whether the patient makes maximum or minimum effort on inspiration. Because the pattern of breathing is unrepresentative of the person's usual pattern of breathing, interpretation of the results obtained by the known apparatus is difficult and may be inaccurate.

It is an aim of the present invention to reduce the above mentioned problem.

Accordingly, in one non-limiting embodiment of the present invention there is provided apparatus for determining respiratory muscle endurance of a person, which apparatus comprises a mouthpiece through which the person inspires, load-providing means for providing a pressure against which the person inspires, and pressure control means for controlling the pressure, the pressure control means being such that it controls the pressure in response to a breathing pattern of the person.

The apparatus of the present invention is able to be operated such that it is regulated according to the person's breathing pattern. This gives more accurate results in the determination of the respiratory muscle endurance of the person.

The apparatus may be one in which the load-providing means is a rotary valve having an orifice which is variable in size. The valve is preferably a rotary valve. The rotary valve is preferably a servo-controlled rotary valve.

The apparatus may be one in which the pressure control means comprises an electronic processor. The apparatus may be one in which the pressure control means controls breathing power in the form of pressure times flow. Alternatively, the apparatus may be one in which the pressure control means controls resistance at the person's mouth in the form of pressure divided by flow. In either instance, the apparatus may be one in which the processor receives a first input in the form of the pressure and a second input in the form of the flow.

The processor may include a display screen. The processor may additionally or alternatively include a hard copy print-out means. Usually, the processor will be a micro-processor.

Preferably, the apparatus is one in which the measurements are obtainable as measurements at the mouthpiece. The measurements may be obtainable as measurements at positions other than the mouthpiece if desired.

Embodiments of the invention will now be described solely by way of example and with reference to the accompanying drawings in which:

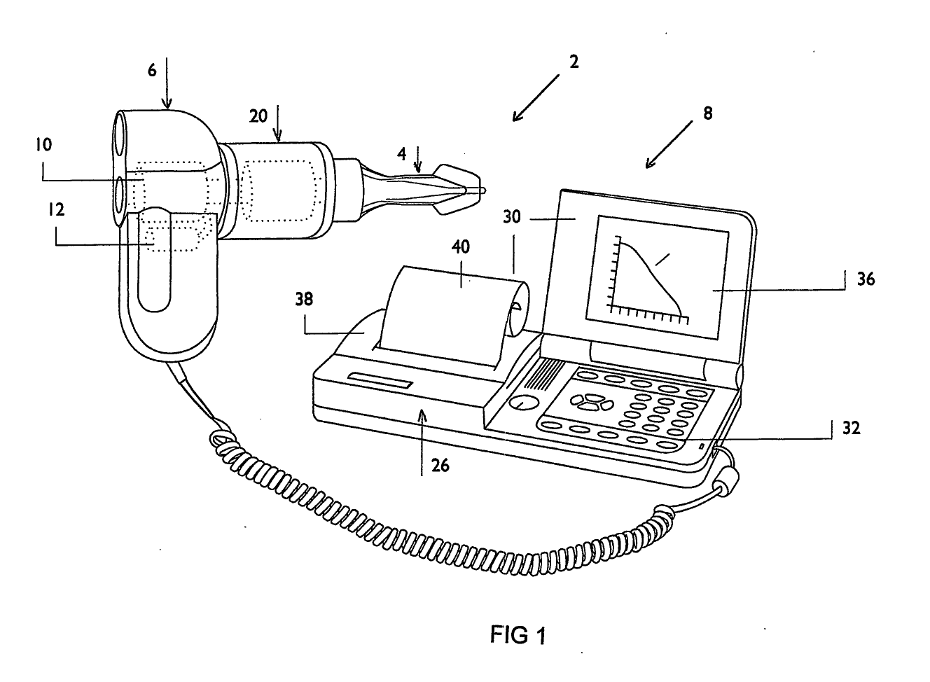

FIG. 1 shows apparatus for determining respiratory muscle endurance of a person;

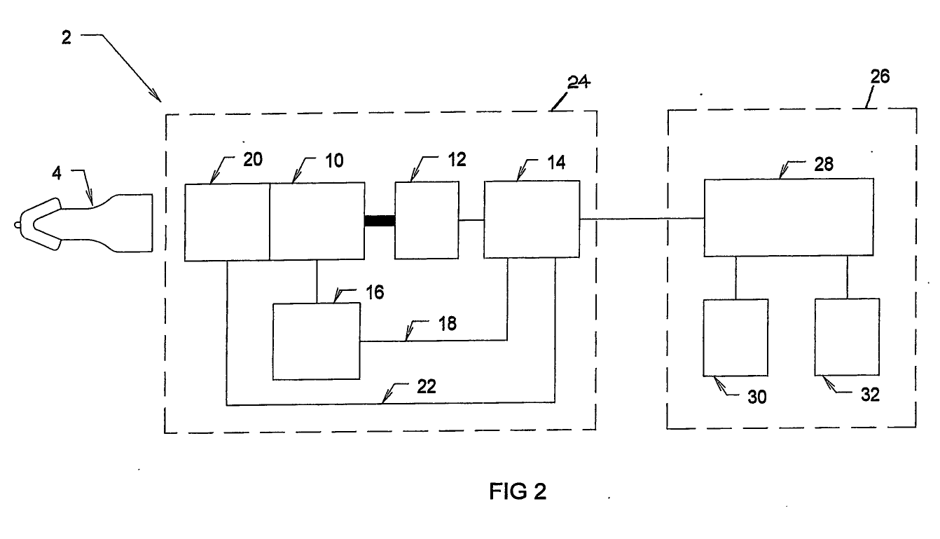

FIG. 2 shows in block diagram form the apparatus shown in FIG. 1;

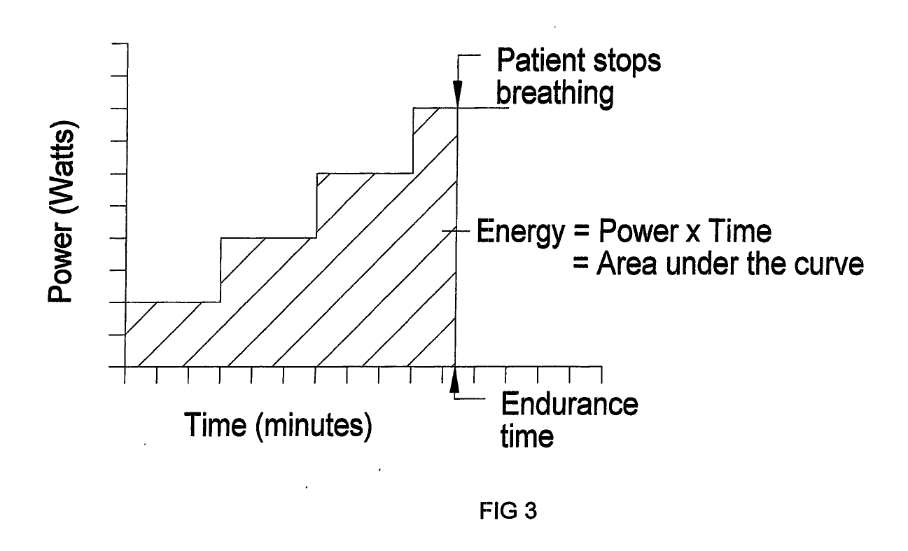

FIG. 3 is a graph showing power plotted against time in order to give energy expended by a person; and

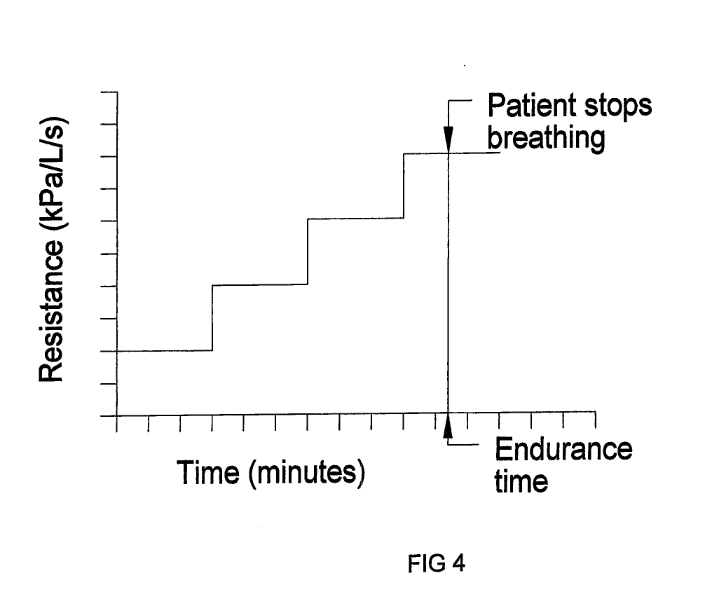

FIG. 4 is a graph showing resistance plotted against time.

Referring to FIGS. 1 and 2, there is shown apparatus 2 for determining respiratory muscle endurance of a person. The apparatus 2 comprises a mouthpiece 4 through which the person inspires. The apparatus 2 also comprises load-providing means 6 for providing a pressure against which the person inspires. The apparatus further comprises pressure control means 8 for controlling the pressure. The pressure control means 8 is such that is controls the pressure in response to a breathing pattern of the person. This enables the breathing power in the form of pressure times flow to be controllable.

The pressure control means 8 comprises a valve 10 having an orifice which is variable in size. The valve 10 is a rotary valve which is servo-controlled by a motor 12. An electronic processor control circuit 14 controls operation of the motor 12. A pressure transducer 16 obtains pressure at the valve 10 and translates this pressure into electrical signals which are fed via a line 18 to the processor control circuit 14. In this way, the processor control circuit 14 is able to be fed with a first input in the form of the pressure at the valve 10.

A flow transducer senses the flow, and appropriate electrical signals are sent along line 22 as a second input for the processor control circuit 14.

As shown in FIG. 2, the parts 10, 12, 14, 16, 18, 20, 22 form a controlled respiratory power transducer 24. The controlled respiratory power transducer 24 is connected to micro-processor control means 26 as shown.

The micro-processor control means 26 comprises a micro-processor circuit 28, display means 30 and a keypad 32. The micro-processor control circuit 28 is shown linked by line 34 to the processor control circuit 14. The processor control circuit 14 may be separate from or part of the micro-processor circuit 28.

FIG. 1 shows the keypad 32. FIG. 1 also shows the display means 30 in the form of a display screen 36 and a printer 38. The printer 38 gives a hard copy print out on paper 40.

FIG. 3 shows power in watts plotted against time in minutes. FIG. 3 also shows energy in the form of power times time which is equal to the area under the curve. FIG. 3 also indicates endurance time and where a patient stops breathing. FIG. 3 illustrates how, in one application, power is able to be progressively increased after a fixed interval of time according to a pre-defined protocol such that the total time taken for the test optimises the discriminatory function of the test for the condition of interest.

The apparatus 2 may alternatively be used to control resistance at a person's mouth, this being in the form of pressure divided by flow. In this connection, FIG. 4 shows resistance plotted against time.

It is to be appreciated that the embodiments of the invention described above with reference to the accompanying drawings have been given by way of example only and that modifications may be effected. Thus, for example, the shape of the mouthpiece 4 may be varied from that shown.

1. Apparatus for determining respiratory muscle endurance of a person, which apparatus comprises a mouthpiece through which the person inspires, load-providing means for providing a pressure against which the person inspires, and pressure control means for controlling the pressure, the pressure control means being such that it controls the pressure in response to a breathing pattern of the person.

2. Apparatus according to claim 1 in which the load-providing means is a rotary valve having an orifice which is variable in size.

3. Apparatus according to claim 2 in which the valve is a rotary valve.

4. Apparatus according to claim 3 in which the rotary valve is a servo-controlled valve.

5. Apparatus according to claim 4 in which the pressure control means comprises an electronic processor, and in which the processor receives a first input in the form of the pressure and a second input in the form of the flow.

6. Apparatus according to claim 5 in which the processor includes a display screen.

7. Apparatus according to claim 5 in which the processor includes a hard copy print-out means.

8. Apparatus according to claim 5 in which the processor is a microprocessor.

9. Apparatus according to claim 1 in which measurements are obtainable as measurements at the mouthpiece.
